(19) United States
(12) Patent Application Publication
(10) Pub. No.: US 2025/0320507 A1
(43) Pub. Date: Oct. 16, 2025

(54) APPLICATION OF ZINC FINGER PROTEIN 217 GENE IN PREPARING MEDICINES FOR TREATING OSTEOPOROSIS

(71) Applicants: Huazhong Agricultural University, Wuhan City (CN); Tongji Hospital, Tongji Medical Collage, Huazhong University of Science & Technology, Wuhan City (CN)

(72) Inventors: Jian PENG, Wuhan City (CN); Hongkui WEI, Wuhan City (CN); Jinxin LU, Wuhan City (CN); Fengjin GUO, Wuhan City (CN); Siwen Jiang, Wuhan City (CN); Yuanfei ZHOU, Wuhan City (CN)

(21) Appl. No.: 19/248,820

(22) Filed: Jun. 25, 2025

Related U.S. Application Data
(63) Continuation of application No. PCT/CN2024/074389, filed on Jan. 29, 2024.

(30) Foreign Application Priority Data
Nov. 28, 2023 (CN) 202311601755.2

(51) Int. Cl. C12N 15/115; A61P 19/10

(52) U.S. Cl. CPC C12N 15/115; A61P 19/10; C12N 2310/14; C12N 2310/16

(57) ABSTRACT

An application of Zfp217 gene in preparing medicines for treating osteoporosis is provided. The present disclosure provides a method for treating osteoporosis by inhibiting an expression of Zfp217 gene. A reagent for inhibiting the expression of Zfp217 gene includes an aptamer for inhibiting the expression of Zfp217 gene in bone marrow mesenchymal stem cells.

### CROSS-REFERENCE TO RELATED APPLICATIONS

This application is a continuation of International Patent Application No. PCT/CN2024/074389, filed Jan. 29, 2024, and claims priority of Chinese Patent Application No. 202311601755.2, filed on Nov. 28, 2023. The entire contents of International Patent Application No. PCT/CN2024/074389 and Chinese Patent Application No. 202311601755.2 are incorporated herein by reference.

### INCORPORATION BY REFERENCE STATEMENT

This statement, made under Rules 77 (b) (5) (ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: SequenceListing.xml
Creation date: Jun. 25, 2025
Byte size: 25,774

### TECHNICAL FIELD

The present disclosure relates to the field of biomedicine, and in particular to an application of a zinc finger protein 217 (Zfp217) gene in preparing medicines for treating osteoporosis.

### BACKGROUND

There is a close relationship between bone development and the health of the organism. Bones provide mechanical support for movement, protect vital organs, and regulate the metabolism of calcium and minerals, and a range of bone-related disorders may arise when bones are poorly formed. Osteoporosis is a growing concern as the population ages and lives longer, and is considered one of the major global epidemics. Age-related estrogen deficiency is the most common cause of osteoporosis, in addition to the absence of mechanical stress. The disease is recognized by a decrease in bone mineral density and an increase in bone marrow fat. Current strategies for the treatment of osteoporosis-related diseases mainly involve inhibiting osteoclast resorption using bisphosphonates and denosumab. However, most of the anti-resorptive drugs used to treat osteoporosis have side effects.

Bone marrow mesenchymal stem cells (BMSCs) play an important role in bone growth and development and are regulated by multiple signals in a sophisticated and complex manner. However, many new and critical transcription factors have yet to be identified in the process of bone formation, and therefore the regulatory network of bone formation needs to be further improved. BMSCs are capable of differentiating into a variety of cell lines, including adipocytes and osteoblasts. However, the molecular mechanisms underlying the transition of BMSCs from osteoblast to adipocyte differentiation are unknown. Therefore, a further understanding of the mechanisms underlying the fate determination of BMSCs is important for exploring the development of novel therapeutic approaches for osteoporosis.

### SUMMARY

The objective of the present disclosure is to provide an application of a Zfp217 gene in preparing medicines for treating osteoporosis, so as to solve the problems existing in the prior art. In the present disclosure, it is experimentally demonstrated that intramedullary injection of BMSCs targeting aptamer-agomir-503-5p/siZfp217 stimulates bone formation and reduces bone marrow fat accumulation, thus providing a new mechanism and a new therapeutic target for age-associated, as well as mechanically stress-deficient, bone loss.

In order to achieve the above objectives, the present disclosure provides the following technical scheme.

The present disclosure provides a method for treating osteoporosis, which includes inhibiting an expression of the Zfp217 gene to ameliorate osteoporosis symptoms.

Optionally, the osteoporosis includes osteoporosis caused by estrogen deficiency and osteoporosis caused by mechanical stress deficiency.

Optionally, by inhibiting the expression of the Zfp217 gene, an expression of a Zfp521 gene is promoted, thereby promoting a formation of osteoblasts.

Optionally, by inhibiting the expression of the Zfp217 gene, bone marrow fat is reduced and bone mass is increased.

The present disclosure also provides a method for regulating a differentiation of BMSCs into osteoblasts or adipocytes, which is realized by regulating the expression of Zfp217 gene, where the BMSCs differentiate into osteoblasts when the expression of Zfp217 gene is inhibited, and the BMSCs differentiate into adipocytes when the expression of the Zfp217 gene is promoted.

The present disclosure also provides a medicine for treating osteoporosis, where the medicine includes a reagent for inhibiting the expression of Zfp217 gene.

Optionally, the reagent for inhibiting the expression of Zfp217 gene includes an aptamer for inhibiting the expression of Zfp217 gene in BMSCs.

Optionally, the aptamer for inhibiting the expression of Zfp217 gene in BMSCs includes a sequence as shown in SEQ ID NO:25.

Optionally, the osteoporosis includes osteoporosis caused by estrogen deficiency and osteoporosis caused by mechanical stress deficiency.

The present disclosure achieves the following technical effects.

The present disclosure demonstrates for the first time experimentally that inhibition of Zfp217 gene expression alleviates bone loss caused by estrogen deficiency or mechanical stress deficiency. The present disclosure also reveals that Zfp217 negatively regulates osteoblast differentiation and positively regulates adipogenesis in part by competitively binding to the Zfp521 promoter with ERα, thereby inhibiting the expression of Zfp521, causing the differentiation of mesenchymal stem cells to adipose tissues, and ultimately reducing bone formation and increasing adipogenesis. The present disclosure provides a new mechanism and a new therapeutic target for the treatment of osteoporosis.

### BRIEF DESCRIPTION OF THE DRAWINGS

FIG. 1A shows the mRNA expression levels of Zfp217 in young (average age: 23 years old) and old (average age: 67 years old) donor bone samples detected by qRT-PCR.

FIG. 1B shows the mRNA expression levels of adipogenic gene C/EBPα and PPARγ in young (average age: 23 years old) and old (average age: 67 years old) donor bone samples detected by qRT-PCR.

FIG. 1C shows the mRNA expression levels of Runx2, Osx, Opn and Ocn in young (average age: 23 years old) and old (average age: 67 years old) donor bone samples detected by qRT-PCR.

FIG. 2A shows the tissue expression profile of one-month-old wild mouse Zfp217 during osteogenic differentiation of mesenchymal stem cells.

FIG. 2B shows the tissue expression profile of three-month-old wild mouse Zfp217 during osteogenic differentiation of mesenchymal stem cells.

FIG. 2C shows the expression pattern of Zfp217 detected by qPCR on days 0, 3, 7, 10, 14, 18, and 21 of osteogenic induction of mesenchymal stem cells C3H10T1/2.

FIG. 3A is a 3D reconstructed image of cancellous bone at the distal femur of mice aged 1 month scanned by micro-CT.

FIG. 3B shows the quantitative analysis of bone volume fraction (BV/TV) of mice aged 1 month.

FIG. 3C shows the quantitative analysis of average number of cancellous bone (Tb.N) of mice aged 1 month.

FIG. 3D shows the quantitative analysis of average thickness of cancellous bone (Tb.Th) of mice aged 1 month.

FIG. 3E shows the quantitative analysis of average spacing of cancellous bone (Tb. Sp) of mice aged 1 month.

FIG. 3F is a 3D reconstructed image of cancellous bone at the distal femur of mice aged 3 months scanned by micro-CT.

FIG. 3G shows the quantitative analysis of BV/TV of mice aged 3 months.

FIG. 3H shows the quantitative analysis of Tb.N of mice aged 3 months.

FIG. 3I shows the quantitative analysis of Tb.Th of mice aged 3 months.

FIG. 3J shows the quantitative analysis of Tb.Sp of mice aged 3 months.

FIG. 4A is a 3D reconstructed image of the cortical bone of the distal femur of 1-month-old mice scanned by micro-CT.

FIG. 4B shows the quantitative analysis of the average thickness (Cb.Th) of the cortical bone of 1-month-old mice.

FIG. 4C is a 3D reconstructed image of the cortical bone of the distal femur of 3-month-old mice scanned by micro-CT.

FIG. 4D shows the quantitative analysis of the Cb.Th of the cortical bone of 3-month-old mice.

FIG. 5A shows the femur length of wild-type mice and Zfp217^{+/-} type mice aged 1 month measured by vernier caliper.

FIG. 5B shows the bone length of wild-type mice and Zfp217^{+/-} type mice aged 1 month measured by vernier caliper.

FIG. 5C shows the femur length of wild-type mice and Zfp217^{+/-} type mice aged 3 months measured by vernier caliper.

FIG. 5D shows the bone length of wild-type mice and Zfp217^{+/-} type mice aged 3 months measured by vernier caliper.

FIG. 6A shows the Safranin fixation green staining of tibia of wild-type mice and Zfp217^{+/-} type mice aged 1 month.

FIG. 6B shows the Safranin fixation green staining of tibia of wild-type mice and Zfp217^{+/-} type mice aged 3 months.

FIG. 7A shows the detection of OCN levels in serum of mice aged 1 month by ELISA.

FIG. 7B shows the detection of OCN levels in serum of mice aged 3 months by ELISA.

FIG. 8A shows the key genes of bone formation in wild-type mice and Zfp217^{+/-} type mice aged 1 month detected by qPCR.

FIG. 8B shows the key genes of lipogenesis in wild-type mice and Zfp217^{+/-} type mice aged 3 months detected by qPCR.

FIG. 9A shows the detection of the effect of Zfp217 on osteogenic differentiation of C3H10T1/2 cells for alkaline phosphatase and alizarin red staining.

FIG. 9B shows the alkaline phosphatase and alizarin red staining to detect the effect of Zfp217 knockout on osteogenic differentiation of primary mouse mesenchymal stem cells.

FIG. 9C shows qPCR detection of mRNA levels of Zfp217 for osteogenesis/adipogenesis in C3H10T1/2 cells and mouse BMSCs.

FIG. 9D shows qPCR detection of mRNA levels of Runx2 for osteogenesis/adipogenesis in C3H10T1/2 cells and mouse BMSCs.

FIG. 9E shows qPCR detection of mRNA levels of Osx for osteogenesis/adipogenesis in C3H10T1/2 cells and mouse BMSCs.

FIG. 9F shows qPCR detection of mRNA levels of Ocn for osteogenesis/adipogenesis in C3H10T1/2 cells and mouse BMSCs.

FIG. 9G shows qPCR detection of mRNA levels of Opn for osteogenesis/adipogenesis in C3H10T1/2 cells and mouse BMSCs.

FIG. 9H shows qPCR detection of mRNA levels of C/EPBα for osteogenesis/adipogenesis in C3H10T1/2 cells and mouse BMSCs.

FIG. 9I shows qPCR detection of mRNA levels of PPARγ for osteogenesis/adipogenesis in C3H10T1/2 cells and mouse BMSCs.

FIG. 9J shows qPCR detection of mRNA levels of Zfp217 for osteogenic/adipogenic genes in C3H10T1/2 cells and mouse BMSCs after Zfp217 knockout.

FIG. 9K shows qPCR detection of mRNA levels of Runx2 for osteogenic/adipogenic genes in C3H10T1/2 cells and mouse BMSCs after Zfp217 knockout.

FIG. 9L shows qPCR detection of mRNA levels of Osx for osteogenic/adipogenic genes in C3H10T1/2 cells and mouse BMSCs after Zfp217 knockout.

FIG. 9M shows qPCR detection of mRNA levels of Ocn for osteogenic/adipogenic genes in C3H10T1/2 cells and mouse BMSCs after Zfp217 knockout.

FIG. 9N shows qPCR detection of mRNA levels of Opn for osteogenic/adipogenic genes in C3H10T1/2 cells and mouse BMSCs after Zfp217 knockout.

FIG. 9O shows qPCR detection of mRNA levels of C/EPBα for osteogenic/adipogenic genes in C3H10T1/2 cells and mouse BMSCs after Zfp217 knockout.

FIG. 9P shows qPCR detection of mRNA levels of PPARγ for osteogenic/adipogenic genes in C3H10T1/2 cells and mouse BMSCs after Zfp217 knockout.

FIG. 10A is the representative image of micro-CT reconstruction of distal femur.

FIG. 10B is the Safranin fixation green staining of the femurs of WT-SHAM, WT-OVX, Zfp217^{+/-}-Sham and Zfp217^{+/-}-OVX mice, with the scale of 0.5 μm.

FIG. 10C is the quantitative analysis of micro-CT in the epiphyseal region of the distal femur (n=8).

FIG. 10D is the serum OCN levels analyzed by ELISA.

FIG. 10E shows qPCR determination of transcript abundance of key osteogenic gene Zfp217, Runx2, Osx, OCN and OPN in WT-SHAM, WT-OVX, Zfp217^{+/-}-SHAM and Zfp217^{+/-}-OVX mice.

FIG. 10F shows qPCR determination of transcript abundance of key adipogenic gene C/EBPα and PPARγ in WT-SHAM, WT-OVX, Zfp217^{+/-}-SHAM and Zfp217^{+/-}-OVX mice.

FIG. 11A shows the effect of alkaline phosphatase and alizarin red staining to detect Zfp217 on alkaline phosphatase and calcium nodule formation in C3H10T1/2 cells in the presence and absence of estrogen.

FIG. 11B shows the effect of alkaline phosphatase and alizarin red staining to detect Zfp217 and ERα on alkaline phosphatase and calcium nodule formation in C3H10T1/2 cells.

FIG. 11C shows the effects of Zfp217 on key osteogenic genes, such as Runx2, Osx, Ocn, Opn and Zfp521 (n=3), with and without estrogen.

FIG. 11D shows the effects of Zfp217 and ERα on key genes of osteogenesis/adipogenesis by qPCR (n=3).

FIG. 12A shows the expression of Zfp521 in young and old donor bone samples;

FIG. 12B shows the expression of Zfp521 in femurs of wild-type mice and Zfp217^{+/-} type mice aged 1 month and 3 months after Zfp217 was knocked out in vivo, and the expression of Zfp521 in C3H10T1/2 cells after interference/overexpression.

FIG. 12C shows the expression of Zfp521 in C3H10T1/2 cells after interference/overexpression of ERα.

FIG. 12D shows the expression of Zfp521 and Alp in C3H10T1/2 cells after overexpression of ERα and Zfp217.

FIG. 12E shows the protein level of Zfp521 detected by western blot after overexpression of ERα and Zfp217 in C3H10T1/2 cells.

FIG. 13A shows that Zfp217 competes with ERα to bind Zfp521 promoter and inhibit the expression of Zfp521.

FIG. 13B shows the influence of overexpression of Zfp217 and ERα on the activity of Zfp521 promoter in the presence and absence of estrogen.

FIG. 13C shows the binding of Zfp217 and ERα to the Zfp521 promoter detected by ChIP-PCR.

FIG. 13D shows the protein level of ERα and Zfp217 by western blot in the cytoplasm and the nucleus of in the presence and absence of estrogen.

FIG. 13E shows the cell localization of ER and Zfp217 by the method of immunofluorescence in the presence and absence of estrogen.

FIG. 14A shows the effects of Zfp217 on mRNA m⁶A.

FIG. 14B shows that the effects of Zfp217 on Zfp521mRNA m⁶A.

FIG. 14C shows the effects of Zfp217 and Mett13 on mRNA m⁶A.

FIG. 14D shows the interaction between Zfp217 and Mett13 detected by CoIP.

FIG. 15A shows the effects of Zfp217 and Mett13 on the expression of Zfp521.

FIG. 15B shows that MeRIP-qPCR detects the influence of Zfp217 on Zfp521 m⁶A.

FIG. 15C shows the effect of Zfp217 on the RNA stability of zfp521.

FIG. 15D shows the effect of Zfp217 on Zfp521 protein detected by Western blot.

FIG. 16A shows the effect of alkaline phosphatase and alizarin red staining to detect Zfp217 and Zfp521 on alkaline phosphatase and calcium nodule formation in C3H10T1/2 cells in the RNA interference of Zfp217 or RNA interference of Zfp217 and Zfp521.

FIG. 16B shows qPCR determination of transcript abundance of key osteogenic/adipogenic gene Runx2, Osx, C/EBPα and PPARγ in C3H10T1/2 cells in the RNA interference of Zfp217 or RNA interference of Zfp217 and Zfp521.

FIG. 16C shows the effect of alkaline phosphatase and alizarin red staining to detect Zfp217 and Zfp521 on alkaline phosphatase and calcium nodule formation in C3H10T1/2 cells in the overexpression of Zfp217 or overexpression of Zfp217 and Zfp521.

FIG. 16D shows qPCR determination of transcript abundance of key osteogenic/adipogenic gene Runx2, Osx, C/EBPα and PPARγ in C3H10T1/2 cells in the overexpression of Zfp217 or overexpression of Zfp217 and Zfp521.

FIG. 17A shows qPCR analysis of the expression level of Zfp217 in bone marrow mesenchymal stem cells of mice injected with aptamer.

FIG. 17B is a 3D reconstructed image of cancellous bone at the distal femur of the WT-SHAM, OVX, OVX-aptamer agomiR-NC, OVX-aptamer agomiR-503 mice through the caudal vein scanned by micro-CT.

FIG. 17C shows the quantitative analysis of BV/TV, Tb.N, Tb.Th, Tb. Sp and Cb.Th of the WT-SHAM, OVX, OVX-aptamer agomiR-NC, OVX-aptamer agomiR-503 mice.

FIG. 17D is a representative μCT image and adipocyte volume of tibia.

FIG. 17E shows the Safranin fixation green staining of tibia in the WT-SHAM, OVX, OVX-aptamer agomiR-NC, OVX-aptamer agomiR-503 mice.

FIG. 17F shows the quantitative analysis of Ob.N/BS and adipose/tissue volume of the WT-SHAM, OVX, OVX-aptamer agomiR-NC, OVX-aptamer agomiR-503 mice.

FIG. 17G is an ELISA analysis of serum Ocn level.

FIG. 17H shows the mRNA expression of key adipogenic gene C/EBPα and PPARγ of tibia in the WT-SHAM, OVX, OVX-aptamer agomiR-NC, OVX-aptamer agomiR-503 mice.

FIG. 17I shows the mRNA expression of key osteogenic gene Runx2, Osx, Ocn and Opn of tibia in the WT-SHAM, OVX, OVX-aptamer agomiR-NC, OVX-aptamer agomiR-503 mice.

FIG. 17J shows the mRNA expression of Zfp521.

### DETAILED DESCRIPTION OF THE EMBODIMENTS

A number of exemplary embodiments of the present disclosure will now be described in detail, and this detailed description should not be considered as a limitation of the present disclosure, but should be understood as a more detailed description of certain aspects, characteristics and embodiments of the present disclosure.

The main materials tested in the embodiments of the present disclosure are as follows.

### 1. Cell Materials

Mouse C3H10T1/2 mesenchymal stem cells are purchased from the cell bank of China Center for Type Culture Collection (CCTCC) of Chinese Academy of Sciences. Primary mouse BMSCs.

### 2. Experimental Animals

The clean grade C57BL/6 mice used in the experiment and the service of producing Zfp217-knockout mice by prokaryotic injection are provided by Cyagen Biosciences (Guangzhou) Inc.

Mice are raised in the experimental animal center of Huazhong Agricultural University, and all procedures are in accordance with the guidelines of the animal feeding and use committee of Huazhong Agricultural University.

### 3. Bone Specimens

The young (average age: 23 years old) and old (average age: 67 years old) donor bone specimens used in the experiment are from bone surgery patients in Tongji Hospital affiliated to Tongji Medical College of Huazhong University of Science & Technology from 2022 to 2023.

### Embodiment 1

In this embodiment, the inhibitory effect of Zfp217 on the differentiation of mesenchymal stem cells into osteoblasts is revealed. Studies have shown that the overall knock-down of Zfp217 gene may reduce bone loss and fat deposition caused by estrogen deficiency in vivo. In vitro experiments suggest that Zfp217 promotes the adipogenic differentiation of BMSCs, while inhibiting their osteogenic differentiation. In addition, estrogen competes with Zfp217 through estrogen receptor to bind to Zfp521 promoter, thus reducing the inhibitory effect of Zfp217 on Zfp521. In osteoporosis caused by estrogen deficiency, bone formation is stimulated and bone fat accumulation is reduced by injecting targeting aptamer-agomir-503-5p/siZfp217 of BMSCs into bone marrow. Therefore, this embodiment provides a new mechanism and a new therapeutic target for age-related bone loss.

In this embodiment, real-time fluorescence quantitative PCR is used to detect the expression level of each gene, and the related gene primer sequences are shown in Table 1.

According to 2-^{ΔΔCt} algorithm, the relative expression is calculated, in which β-actin is used as internal reference.

TABLE 1
Primer sequences of different genes

| Gene | Primer | Sequence (5′-3′) | SEQ ID NO. |
|---|---|---|---|
| Zfp217 | F | AGAACGGCAGCAGCAAGT | 1 |
| | R | AGTGTGGTGAGAAGGCAGTC | 2 |
| C/EBPα | F | AGCCAAGAAGTCGGTAGA | 3 |
| | R | CGGTCATTGTCACTGGTC | 4 |
| PPARγ | F | TGGGTGAAACTCTGGGAGATTC | 5 |
| | R | AGAGGTCCACAGAGCTGATTCC | 6 |
| Runx2 | F | GCCGGGAATGATGAGAACTA | 7 |
| | R | GGACCGTCCACTGTCACTTT | 8 |
| Osx | F | GCAAGGCTTCGCATCTGA | 9 |
| | R | CAAGTGGTCGCTTCTGGTAA | 10 |
| Ocn | F | ATGGCTTGAAGACCGCCTAC | 11 |
| | R | AGGGCAGAGAGAGAGGACAG | 12 |
| Opn | F | GACGATGATGACGATGATGATGA | 13 |
| | R | TAGGGACGATTGGAGTGAAAGT | 14 |
| β-actin | F | GGCACCACACCTTCTACAATG | 15 |
| | R | GGGGTGTTGAAGGTCTCAAAC | 16 |
| Zfp521 | F | CTCAGCAGACCTCCGATAT | 17 |
| | R | GTAAGACCTCCAAGCAATACT | 18 |

### Test Content

### 1. Expression of Zfp217 and Osteogenic/Adipogenic Genes During Human Bone Aging

In order to study the role of Zfp217 in the differentiation of BMSCs, the expression of Zfp217 in bone samples of young (average age: 23 years old) and old (average age: 67 years old) donors is detected by real-time fluorescence quantitative PCR with β-actin as internal reference gene.

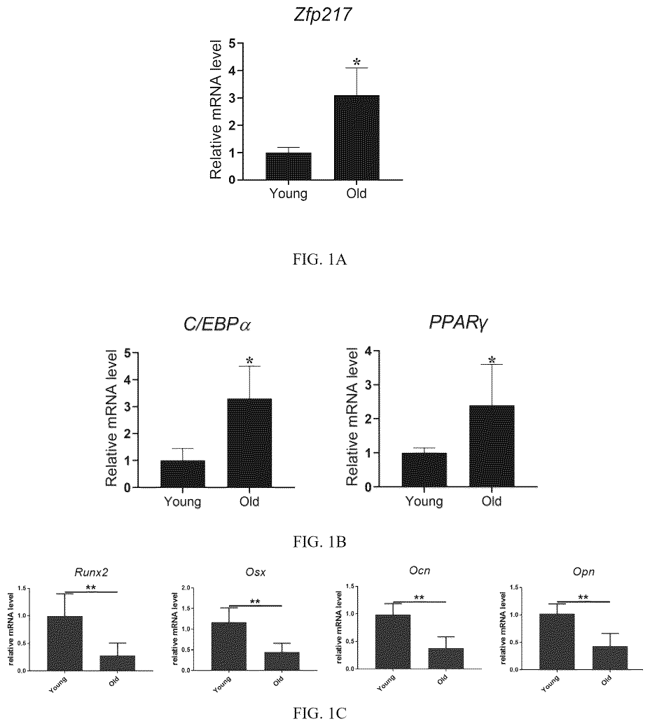

The results show that the expression of Zfp217 in elderly donor bone specimens is increased significantly (FIG. 1A). Then the expression of bone/lipogenesis related genes in young and old donors is studied, and the results show that compared with young donors, the mRNA levels of adipogenesis-related genes (such as C/EBPα and PPARγ) in old donor bone samples are significantly increased (FIG. 1B). At the same time, compared with young donors, the contents of osteogenesis-related genes such as Runx2, Osx, Ocn and Opn in old donor bone samples are significantly decreased (FIG. 1C). To sum up, these results reveal that the expression of Zfp217 in human bones increases with the decrease of bone formation and the increase of fat production.

### 2. Expression Pattern of Zfp217 in the Process of Bone Formation and Osteoblast Differentiation in Mice

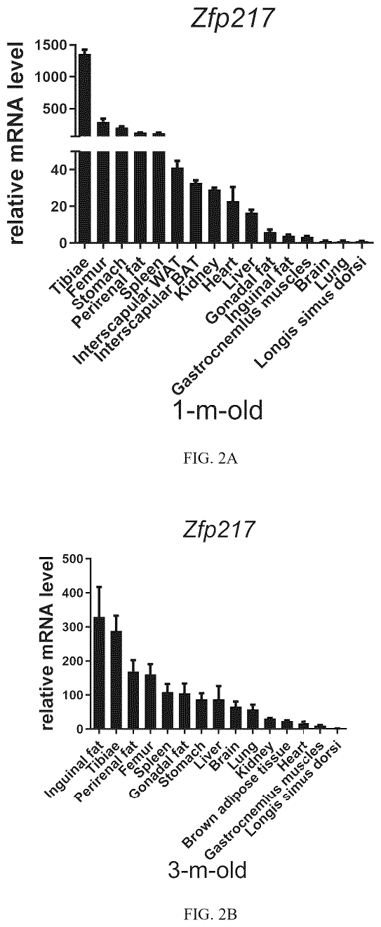

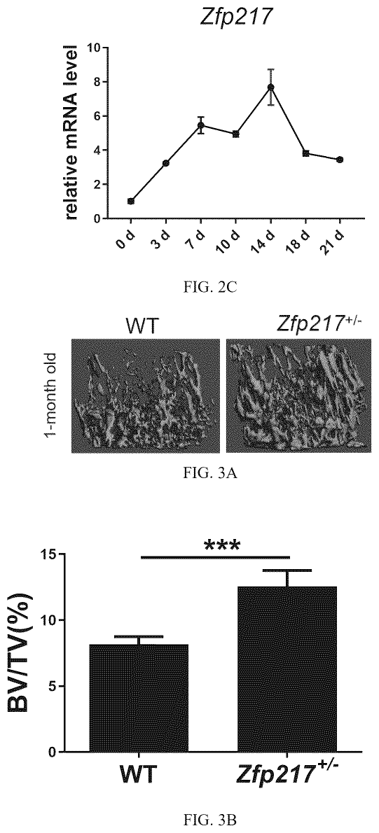

In order to further explore the influence of Zfp217 on bone formation, the heart, liver, spleen, lung, kidney, stomach, brain, gastrocnemius, longissimus dorsi, brown fat, white fat between scapula, perirenal fat, gonadal fat, inguinal fat, femur and tibia of wild-type mice aged 1 month and 3 months are collected respectively, and the temporal and spatial expression spectrum of Zfp217 are detected by qPCR. The results show that Zfp217 is highly expressed in the femur and tibia of 1-month-old and 3-month-old mice, suggesting that Zfp217 may play an important role in the process of bone formation (FIG. 2A and FIG. 2B). After that, mesenchymal stem cells C3H10T1/2 are cultured in osteogenic induction medium (DMEM+Dexamethasone+Vitamin C+β-glycerophosphate), and the mRNA expression pattern of Zfp217 is detected by qPCR at 0, 3, 7, 10, 14, 18 and 21 days after culture, and the results show that in the process of osteogenic differentiation, the mRNA level of Zfp217 increases first and then decreases (FIG. 2C), suggesting that Zfp217 may play an important role in the process of osteogenic differentiation.

### 3. Zfp217 Knockout Causing Increased Bone Mass and Decreased Bone Marrow Fat

In order to detect the effect of Zfp217 knockout on bone formation in mice, a Zfp217 whole-body knockout mouse is constructed in this embodiment. Since Zfp217 double knockout is lethal, this embodiment utilizes Zfp217 heterozygous mice (Zfp217^{+/-}) for experiments.

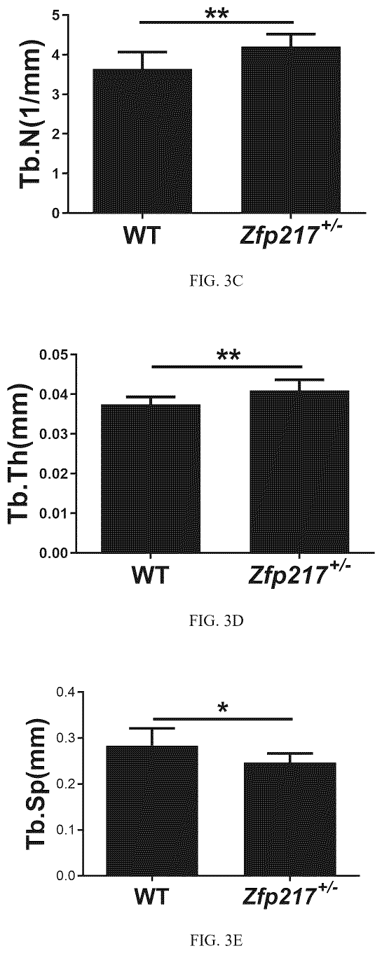

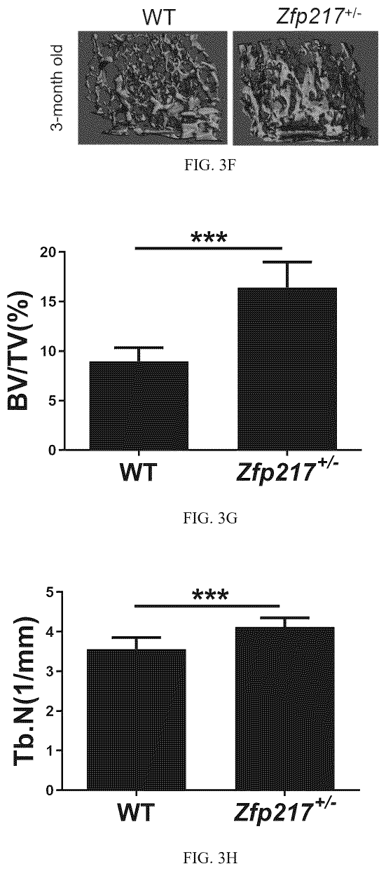

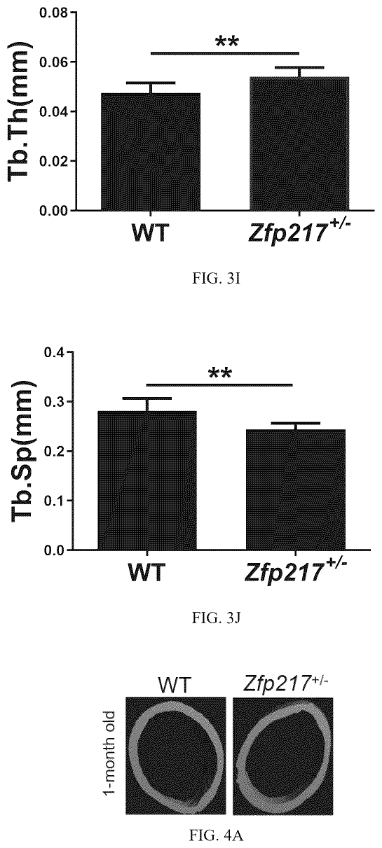

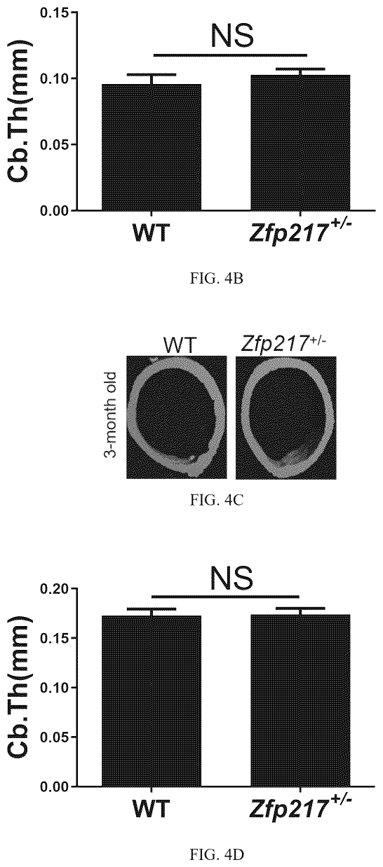

In order to study the influence of Zfp217^{+/-} on the bone formation of mice more intuitively, this embodiment uses micro-CT to analyze the femoral cancellous bone of wild-type mice and Zfp217^{+/-} type mice (1 month old and 3 months old). The results show that the parameters of the cancellous bone of Zfp217^{+/-} type mice are significantly higher than those of wild-type mice. This is manifested by elevated volume fraction of cancellous bone (BV/TV), average thickness of cancellous bone (Tb.Th), and average number of cancellous bones (Tb.N), but reduced average spacing of cancellous bones (Tb.Sp) (FIG. 3A-FIG. 3J). Micro-CT analysis of cortical bone reveals that there is no difference in cortical bone thickness (Ct.Th) between Zfp217^{+/-} type mice and wild-type mice (FIG. 4A-FIG. 4D), suggesting that knockout of Zfp217 promotes cancellous bone formation but has no effect on cortical bone.

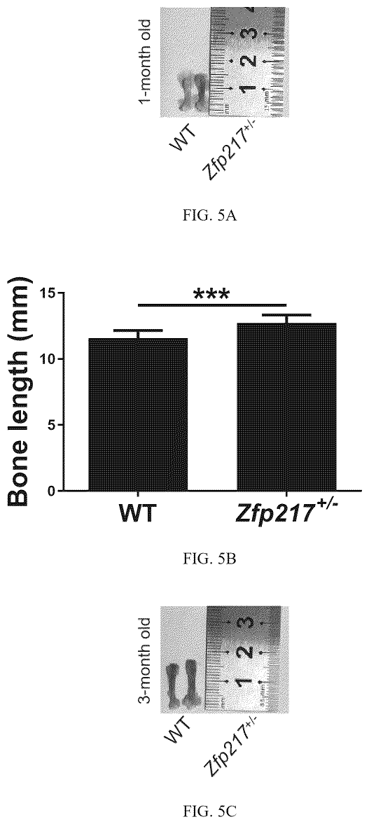

Subsequently, the femur length of wild-type mice and Zfp217^{+/-} type mice (1 month old and 3 months old) is measured with vernier caliper, and it is found that the femur length of Zfp217^{+/-} type mice is significantly longer than that of wild-type mice (FIG. 5A-FIG. 5D).

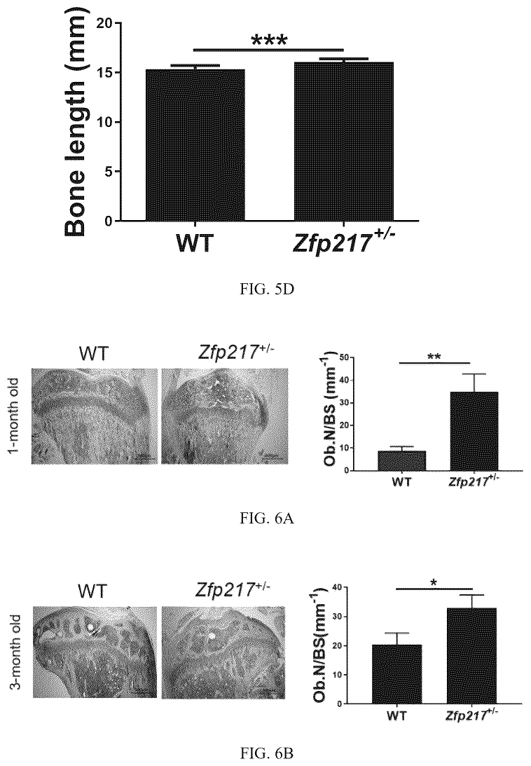

The tibia of wild-type mice and Zfp217^{+/-} type mice aged 1 month and 3 months are detected by Safranin fast green staining. It is found that compared with wild-type mice, the tibia osteoblasts of Zfp217^{+/-} type mice are increased significantly, but the bone marrow fat content has no significant change (FIG. 6A-FIG. 6B).

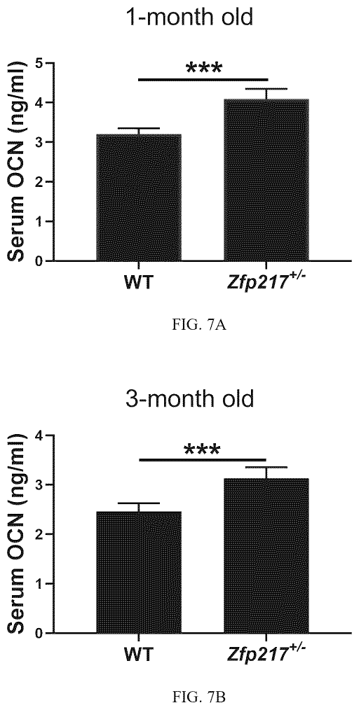

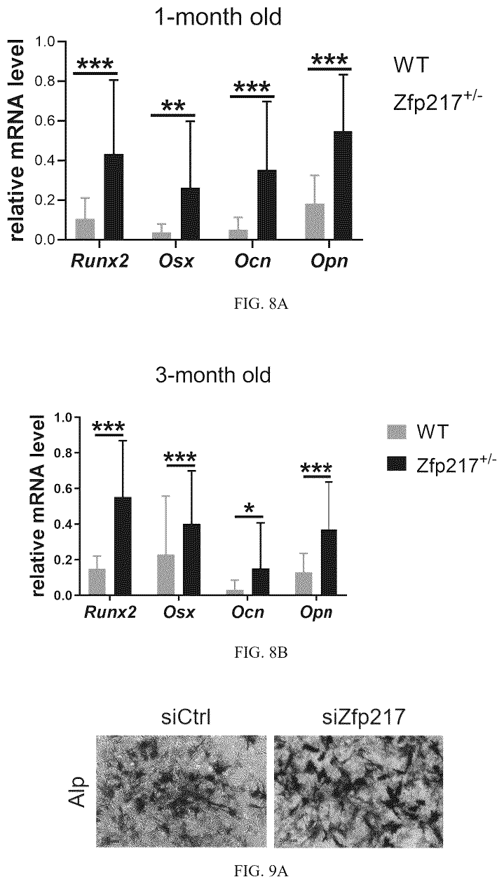

Compared with wild-type mice, the serum bone formation marker OCN of Zfp217^{+/-} type mice is significantly increased as detected by ELISA (FIG. 7A and FIG. 7B). Accordingly, total RNA is extracted from femur, and the expression of genes related to bone and lipogenesis in 1-month-old and 3-month-old mice is detected. The results show that the expression of osteogenesis-related genes Runx2, Osx, Ocn and Opn in 1-month-old and 3-month-old Zfp217^{+/-} mice is higher than that in wild-type mice, while the expression of lipogenesis-related genes C/EBPα and PPARγ is significantly lower than that in wild-type mice (FIG. 8A and FIG. 8B). The above results indicate that the loss of Zfp217 promotes bone formation and reduces bone marrow fat deposition during bone development.

### 4. Control of Mesenchymal Stem Cell Fate Determination by Zfp217 In Vitro

Next, to investigate the role of Zfp217 in osteogenesis and adipogenesis, the effect of Zfp217 on osteogenic and adipogenic differentiation of C3H10T1/2 cells by siRNA and overexpression plasmid-mediated loss-of-function and gain-of-function is investigated.

The construction process of Zfp217 overexpression plasmid is as follows:
according to the CDS sequence of Zfp217 in Gene of http://www.ncbi.nlm.nih.gov/, the website of NCBI (National Center for Biotechnology Informational), primers are designed by using Primer 6.0 software, and the sequences of the primers are as follows:

upstream:
SEQID NO: 19
5′-CGGGATCCATGCCGACCCAGTCCCTCCTC-3′,;
downstream:
SEQID NO: 20
5′-GATATCTCAAGGTTTTTTGTCATTTGGTCGGTAATG-3′,.

The sequences are subsequently amplified from mouse tissue cDNA using the high-fidelity enzyme Pfu, recovered by electrophoresis, ligated into the PCMV-N-flag vector and sequenced, and the Zfp217 overexpression plasmid is obtained by sequence comparison using BLAST (http://blast.ncbi.-nlm.nih.gov/Blast.cgi).

The siRNA target sequences of Zfp217 are:

Zfp217-1:
SEQID NO: 21
5′-CACACTTCCACGGAATCATAC-3′,;
Zfp217-2:
SEQID NO: 22
5′-TCACATCAGCACCTATCTAAC-3′,;
and
negative control (NC):
SEQID NO: 23
5′-TTCTCCGAACGTGTCACGT-3′,.

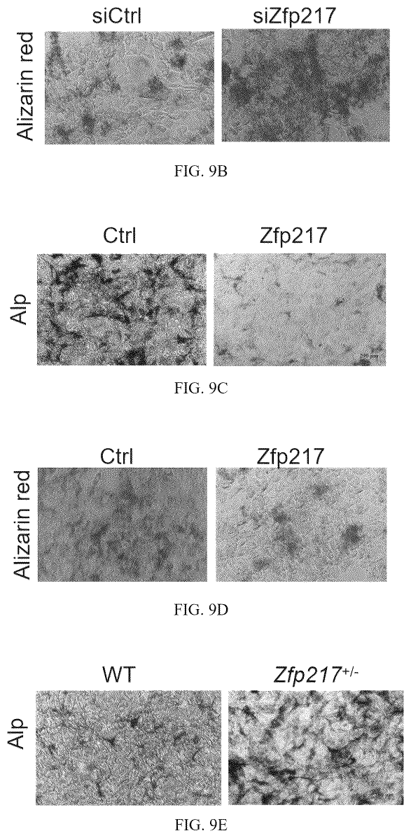

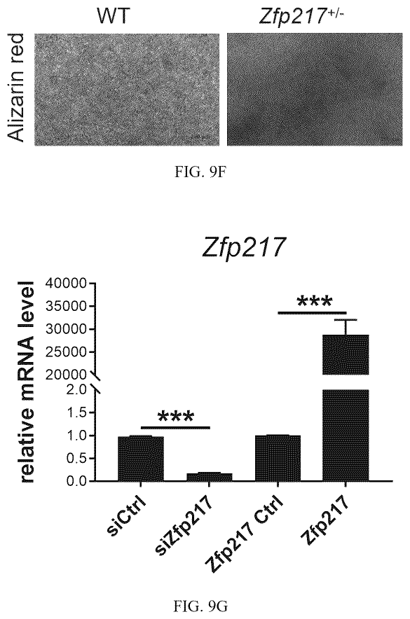

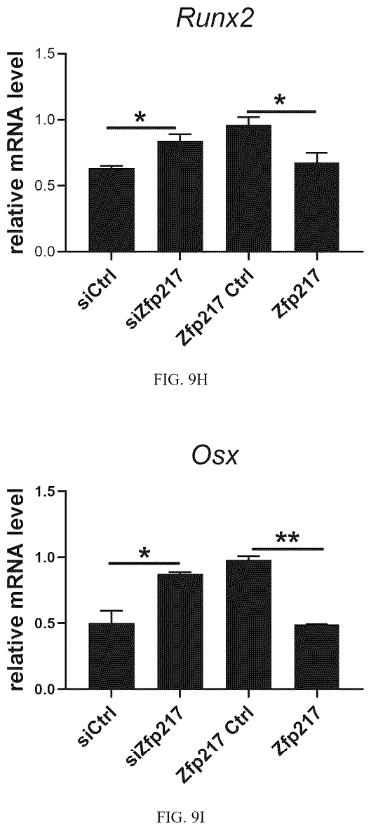

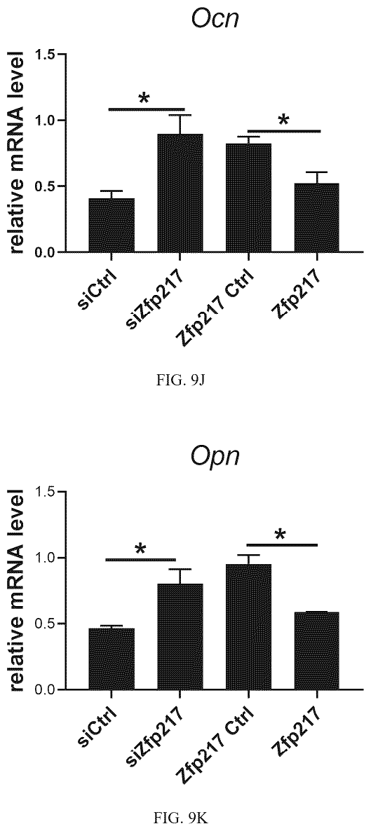

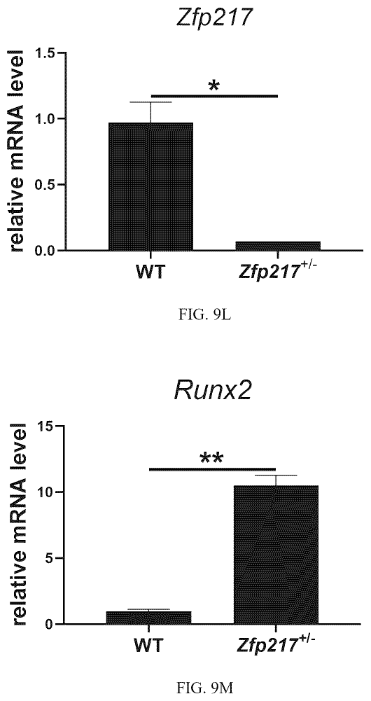

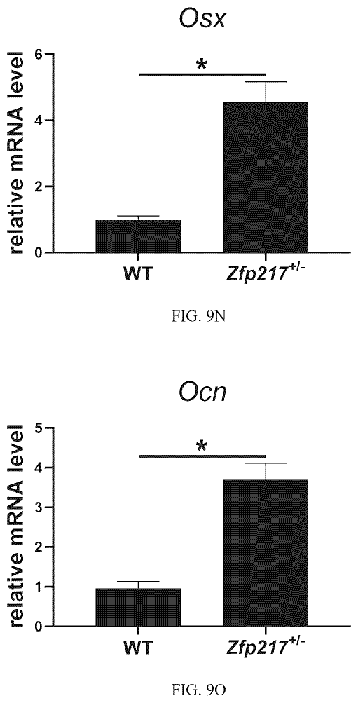

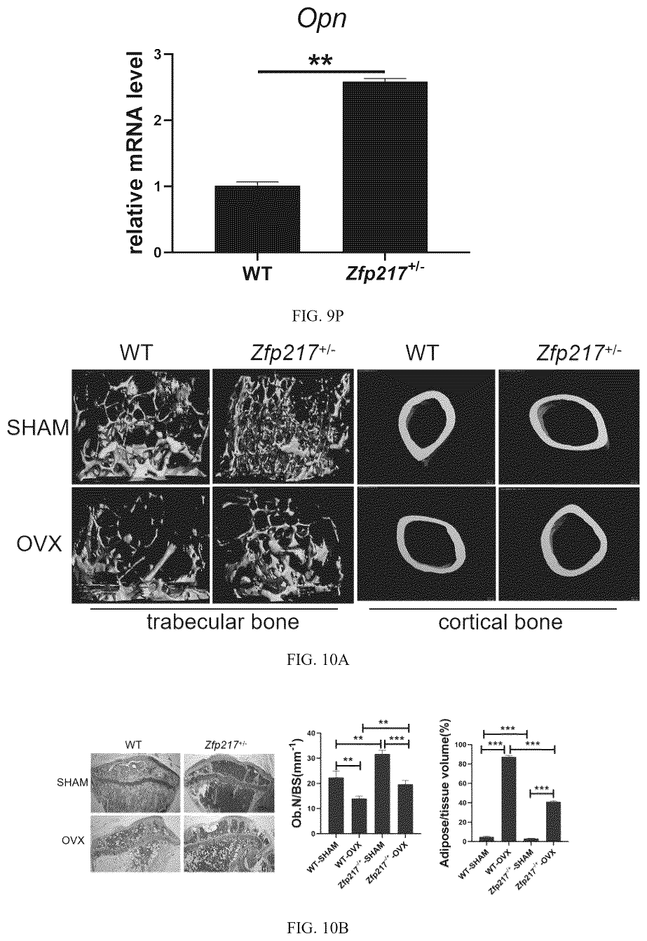

The differentiation of osteoblasts is evaluated by alkaline phosphatase (ALP) staining, alizarin red staining and osteoblast marker mRNA expression. As shown in FIG. 9A-FIG. 9D, interference with Zfp217 significantly increases the number of mineralized nodules stained by ALP and alizarin red. Over-expression of Zfp217 leads to the decrease of ALP staining and the decrease of the number of mineralized nodules. At the same time, interference with Zfp217 significantly increases the mRNA levels of Runx2, Osx, Opn and Ocn, and decreases the expression of C/EBPα and PPARγ. On the contrary, overexpression of Zfp217 significantly decreases the mRNA levels of Runx2, Osx, Opn and Ocn in C3H10T1/2 cells, and increases the expressions of C/EBPα and PPARγ (FIG. 9G-FIG. 9K). Consistent with the results for C3H10T1/2 cells, more ALP staining and mineralized nodules are observed in Zfp217^{+/-} mouse BMSCs than in wild mouse BMSCs (FIG. 9E-FIG. 9F). In addition, the mRNA expressions of the osteoblast markers Runx2, Osx, Opn, and Ocn are increased by Zfp217 knockout, while the expressions of C/EBPα and PPARγ are decreased (FIG. 9L-FIG. 9P). Taken together, these results suggest that Zfp217 promotes lipogenic differentiation while inhibiting osteogenic differentiation of C3H10T1/2 cells and BMSCs.

### 5. Stimulation of Estrogen Deficiency-Induced Osteoporotic Bone Formation and Reduction of Bone Marrow Fat Accumulation by Zfp217 Deficiency

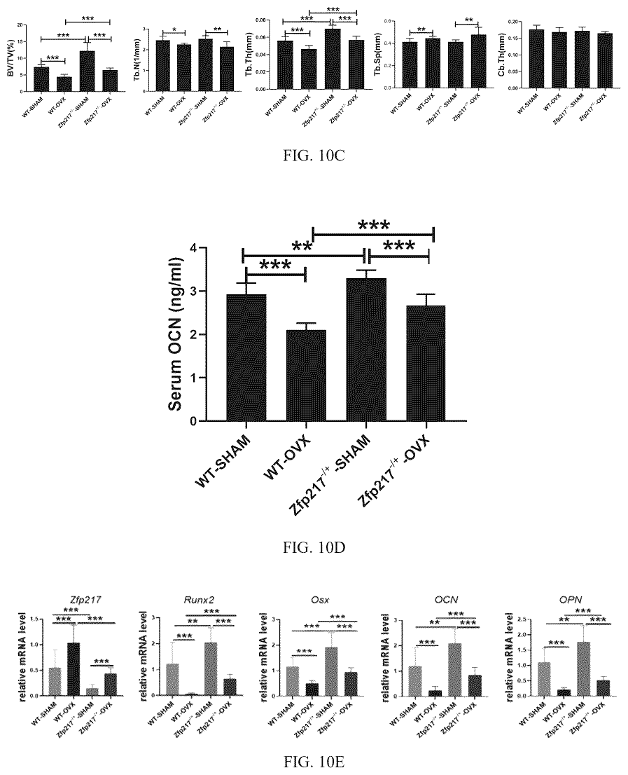

In view of the influence of Zfp217 gene knockout on bone development, whether Zfp217 gene knockout may increase bone mass or reduce lipogenesis to prevent osteoporosis caused by estrogen deficiency is studied in this embodiment. To explore the potential protective role of Zfp217 knockout in osteoporosis, ovariectomy (OVX) is performed using 12-week-old Zfp217^{+/-} and wild-type female mice of the same litter. Ovariectomy simulates bone loss and bone marrow fat accumulation in postmenopausal osteoporosis caused by estrogen deficiency (FIG. 10A-FIG. 10D). Analysis of microCT reveals that in the sham operation group, BV/TV and Tb.Th are significantly increased in Zfp217^{+/-} mice compared to wild-type mice. Compared with WT-OVX mice, BV/TV and Tb.Th of Zfp217^{+/-}OVX mice are also significantly increased, which is consistent with the results of sham operation group. However, in the absence of Zfp217, the changes of Tb.N, Ct.Th and Tb.Sp are not obvious (FIG. 10A and FIG. 10C). Safranin fast green staining clearly reveals a decrease in the number of osteoblasts (Ob.N/BS) and an increase in bone marrow fat after ovariectomy, and these changes are ameliorated after Zfp217 knockdown (FIG. 10B). In addition, compared with WT-OVX mice, the serum Ocn level of Zfp217^{+/-}OVX mice is significantly increased (FIG. 10D). The qPCR analysis also shows that Zfp217^{+/-} mice exhibit significant up-regulation of osteogenic marker genes and significant down-regulation of adipogenic marker genes after OVX compared with WT-OVX mice (FIG. 10E and FIG. 10F). These results indicate that Zfp217 deficiency alleviates estrogen deficiency-induced osteoporosis by promoting bone formation and reducing bone marrow fat accumulation.

### 6. Reversal of the Effect of Estrogen on the Fate Determination of Mesenchymal Stem Cells by Zfp217

In view of the saving effect of Zfp217 knockout on bone quality and bone marrow fat in ovariectomized model, the role of Zfp217 in estrogen signal transduction is explored in this embodiment. Overexpression of Zfp217 in C3H10T1/2 cells with and without the addition of estrogen E2 (MCE, USA, HY-B0141) are cultured in osteogenic induction and differentiation medium for 7 and 14 days, during which the osteogenic induction medium is changed every 3 days.

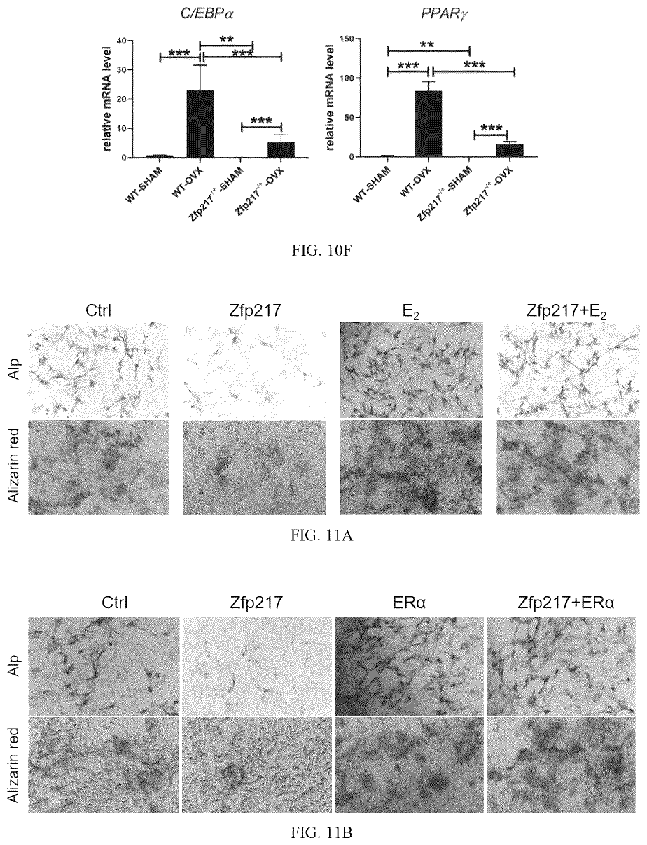

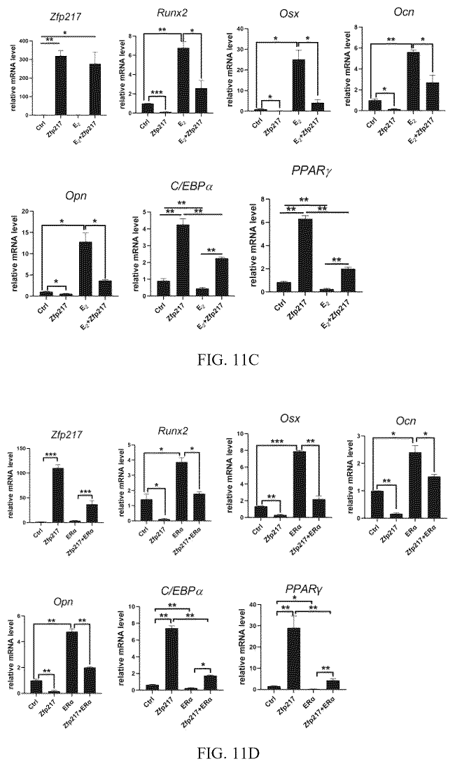

Compared to cells in the control group, E2 treatment shows a significant increase in alkaline phosphatase activity from day 7 after differentiation and more mineralized nodules (FIG. 11A). In C3H10T1/2 cells, E2 treatment with concomitant overexpression of Zfp217 results in decreased alkaline phosphatase activity and calcium mineralization compared to cells with E2 treatment only (FIG. 11A). Compared with the control group, E2 treatment shows increased expression of osteogenic differentiation markers such as Runx2, Osx, Opn and Ocn, and significantly decreased expression of adipose differentiation genes, which are consistent with the differentiation and mineralization experiments. Compared with cells only treated with E2, the simultaneous overexpression of Zfp217 significantly decreases the mRNA levels of Runx2, Osx, Opn and Ocn, and increases the expression of fat-related genes (FIG. 11C).

In this embodiment, overexpression of ERα is found to significantly increase alkaline phosphatase activity and the number of mineralized nodules, while the effect of ERα on osteogenesis is found to be reduced by overexpression of Zfp217 (FIG. 11B). Similarly, overexpression of Zfp217 suppresses the promotion of osteogenic genes by ERα and increases the expression of adipogenic genes (FIG. 11D). In summary, these results indicate that Zfp217 regulates the fate of stem cells in an opposite way to estrogen.

### 7. Inhibition by Zfp217 Against the Promotion of Zfp521 by ERα

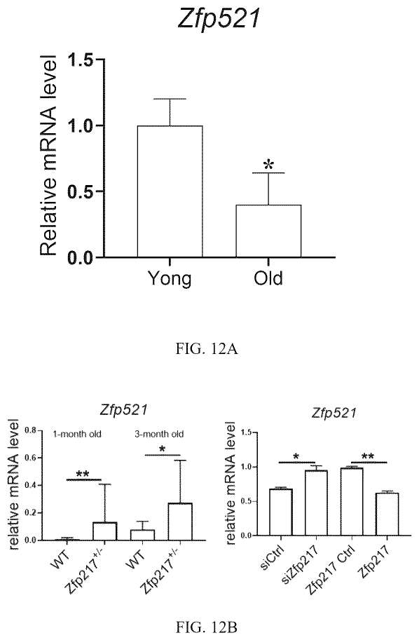

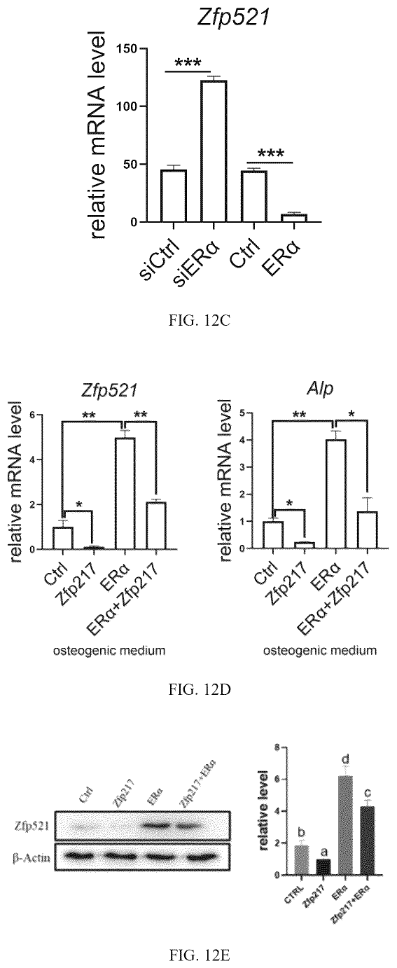

In this embodiment, Zfp521 is predicted to be a target gene of Zfp217 using bioinformatics methods, and the effect of Zfp217 on it is investigated. Primarily, Zfp521 expression is significantly lower in bone specimens from older donors than younger donors (FIG. 12A). In addition, the expression of Zfp521 is significantly higher in the femurs of 1-month-old and 3-month-old Zfp217^{+/-} mice than in wild-type mice. Moreover, overexpression of Zfp217 in C3H10T1/2 cells significantly suppresses Zfp521 expression, whereas interference with Zfp217 results in a significantly higher level of Zfp521 expression (FIG. 12B).

In order to study the role of Zfp217 in estrogen signaling pathway, Zfp217 and ERα are overexpressed in C3H10T1/2 cells to determine the effects of Zfp217 and ERα on target genes. Consistent with the results of C3H10T1/2 cells, the deletion of Zfp217 promoted the expression of Zfp521 in vitro and in vivo (FIG. 12B-FIG. 12C). In addition, the alkaline phosphatase gene (Alp) is an important target of ERα in osteogenic differentiation. Overexpression of Zfp217 inhibits the expression of Zfp521 and Alp, whereas overexpression of ERα increases the expression of Zfp521 and Alp. In addition, overexpression of ERα while overexpressing Zfp217 decreases the expression of Zfp521 and Alp compared to overexpression of ERα alone (FIG. 12D). Meanwhile, it is observed by protein immunoblotting that overexpression of ERα along with overexpression of Zfp217 decreases the protein level of Zfp521 compared to overexpression of ERα alone (FIG. 12E). Overall, Zfp217 reverses the promotion of Zfp521 by ERα.

8. Competition of Zfp217 with ERα for Binding to the Zfp521 Promoter and Repression of Zfp521 Expression

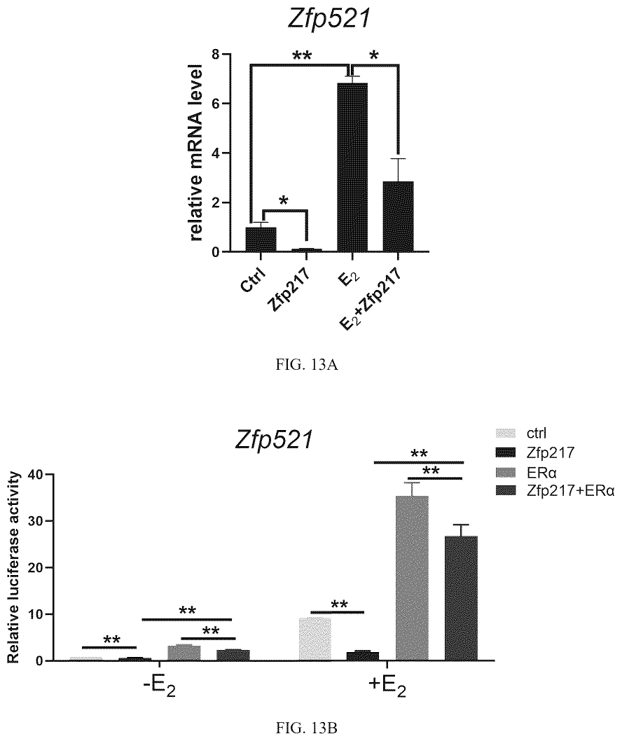

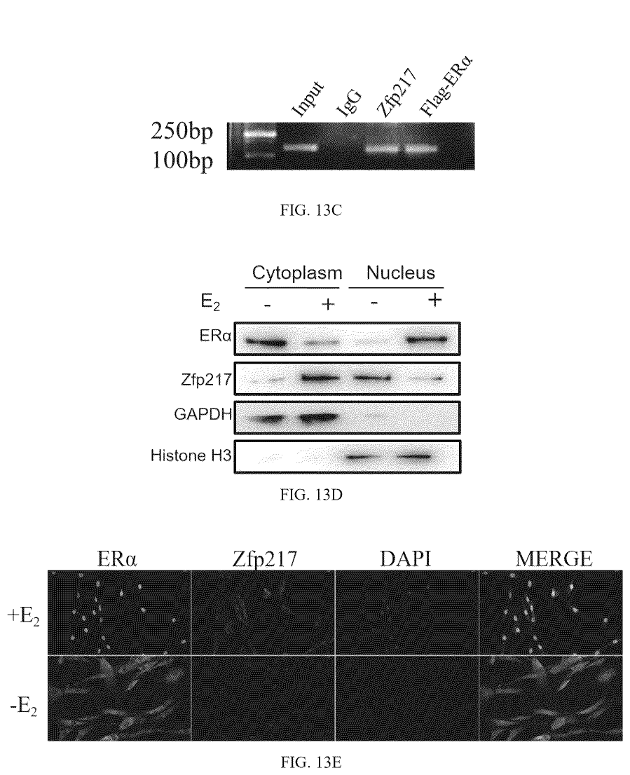

To investigate the regulatory effects of Zfp217 and estrogen on Zfp521, Zfp217 is first overexpressed in the presence and absence of estrogen and the effects on Zfp521 are analyzed. Consistent with previous findings, overexpression of Zfp217 significantly suppresses Zfp521 expression. However, such suppression is effectively alleviated upon administration of estrogen (FIG. 13A).

In order to study the transcriptional regulation mechanism of estrogen and Zfp217 on Zfp521, a double luciferase reporter gene experiment is conducted to study whether Zfp217 and ERα may transcriptionally regulate the promoter of Zfp521 in the presence or absence of estrogen. The results show that ERα significantly promotes the activity of Zfp521 promoter. Meanwhile, overexpression of ERα and Zfp217 lowers the activity of the Zfp521 promoter as compared to overexpression of ERα alone, and these effects may be amplified by estrogen (FIG. 13B). In addition, ChIP-qPCR results demonstrate that both Zfp217 and ERα are capable of binding to the Zfp521 promoter (FIG. 13C).

In order to further clarify the mechanism of Zfp217 and ERα regulating the transcription of Zfp521, the subcellular localization of Zfp217 and ERα in the presence and absence of estrogen is studied. The results show that under the action of estrogen, ER is mainly located in the nucleus, while Zfp217 mainly exists in the cytoplasm. On the contrary, in the absence of estrogen, Zfp217 mainly accumulates in the nucleus, while ERα mainly resides in the cytoplasm (FIG. 13D).

In addition, chromatin immunoprecipitation is used to detect the enrichment of Zfp217 and ERα on Zfp521 promoter in the presence and absence of estrogen. The results suggest that the enrichment ability of Zfp21 to Zfp521 promoter is higher than that of ERα in the absence of estrogen. On the contrary, in the presence of estrogen, ERα is more abundant in the Zfp521 promoter than Zfp217 (FIG. 13E). The above results suggest that estrogen competes with Zfp217 via estrogen receptor a to bind the Zfp521 promoter, which in turn promotes bone formation and inhibits lipid deposition.

9. Interaction of Zfp217 with Mett13 to Regulate the Level of Intracellular m⁶A Modification

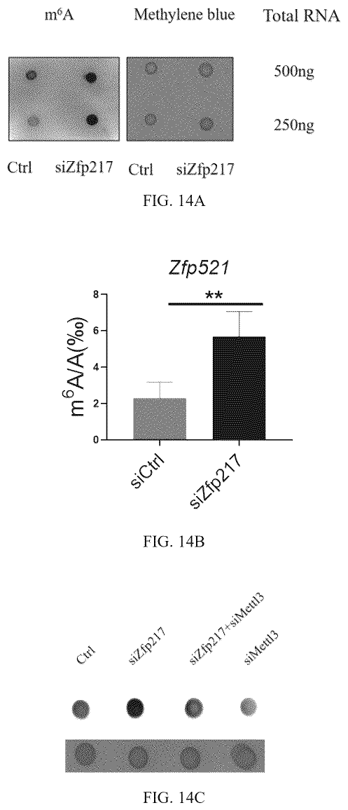

Previous studies have found that Zfp217 is capable of regulating the expression of Zfp521 and then regulating the differentiation of osteoblasts. In order to explore whether Zfp217 is capable of regulating the expression of Zfp521 through m⁶A modification, thus affecting the differentiation of osteoblasts. To investigate whether Zfp217 is capable of regulating the expression of Zfp521 through m⁶A modification and thus affecting osteoblast differentiation, firstly, Zfp217 is interfered upon C3H10T1/2 cells, and then the level of m⁶A modification of mRNAs is detected by spot hybridization and high-performance liquid chromatography-mass spectrometry, and the results show that the level of m⁶A modification of mRNA is significantly increased after interference with Zfp217 (FIG. 14A and FIG. 14B). Then Zfp217 is interfered as well as simultaneously interfering with Zfp217 and Mett13. m⁶A modification levels of mRNAs are significantly increased when interfering Zfp217, and m⁶A modification levels of mRNAs are significantly decreased when interfering with Zfp217 and Mett13 at the same time, as compared to interfering with Zfp217 only (FIG. 14C).

In order to detect whether there is protein interaction between Zfp217 and Mett13 in C3H10T1/2 cells, the protein interaction between Zfp217 and Mett13 is detected in C3H10T1/2 cells by CoIP (FIG. 14D).

### 10. Regulation of Zfp521 Expression by Zfp217 Through Post-Transcriptional Modifications

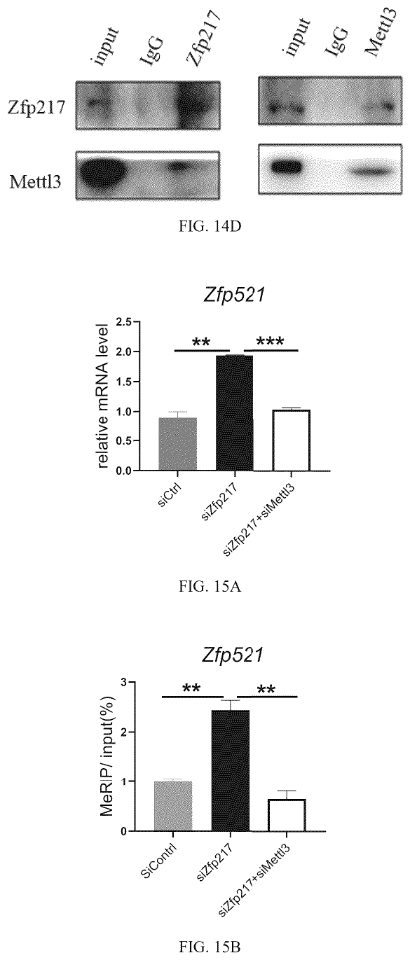

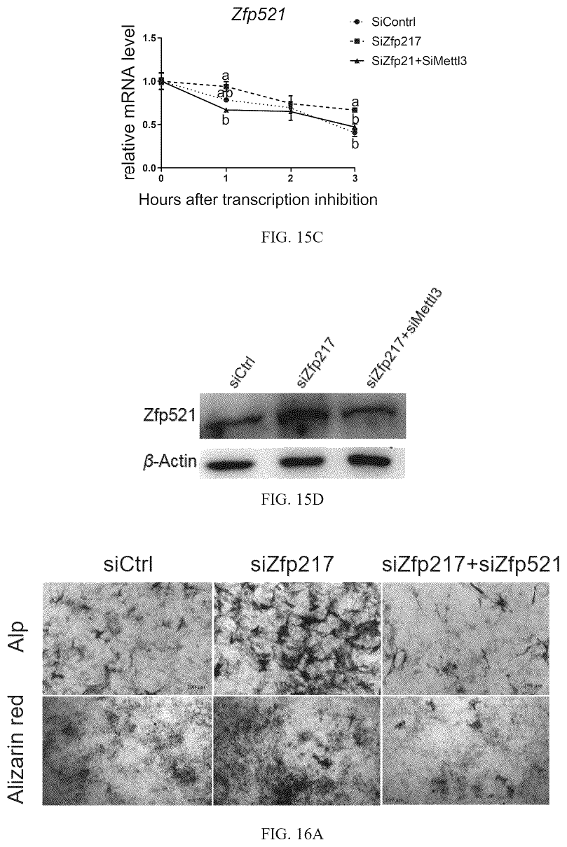

To explore whether Zfp217 is capable of regulating the expression of Zfp521 through Mett13 and thus affecting osteogenic differentiation, this embodiment finds that when Zfp217 is interfered, the expression of Zfp521 increases; and when Zfp217 and Mett13 are interfered at the same time, the expression of Zfp521 decreases compared with that when Zfp217 alone is interfered (FIG. 15A).

Then, by MeRIP-qPCR, it is found that the m⁶A modification on Zfp521 mRNA is increased after interference with Zfp217, while the m⁶A modification on Zfp521 mRNA is decreased when interference with Zfp217 and Mett13 occurred at the same time (FIG. 15B). The mRNA stability analysis reveals that the stability of Zfp521 is elevated after interfering with Zfp217 (FIG. 15C), and by Western blot, the protein expression of Zfp521 is found to be elevated after interfering with Zfp217 (FIG. 15D). Taken together, the above results indicate that Zfp217 regulates the expression of Zfp521 protein by modulating the stability of Zfp521 mRNA.

### 11. Regulation of Mesenchymal Stem Cell Fate Selection by Zfp217 Through Zfp521

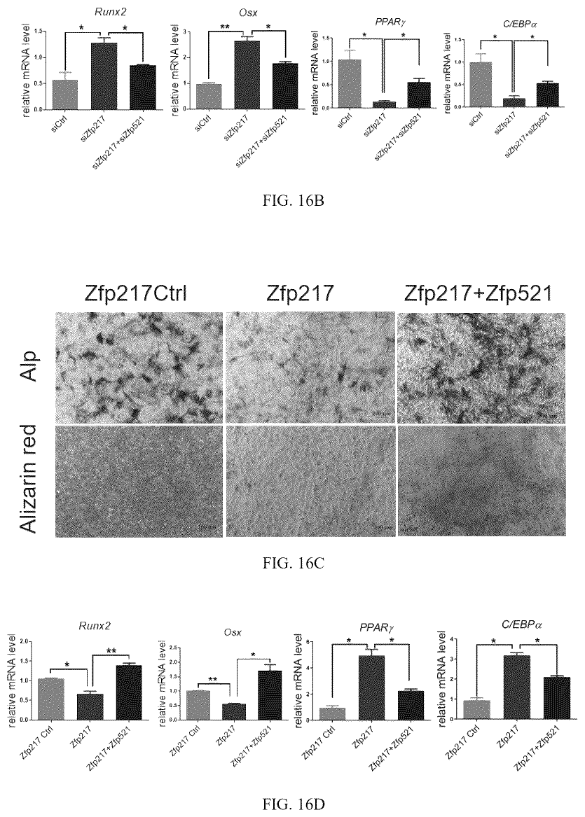

After interfering with Zfp217 on C3H10T1/2 cells, osteogenic differentiation is promoted and lipogenic differentiation is inhibited; whereas when both Zfp217 and Zfp521 are interfered with, osteogenic differentiation is significantly inhibited and lipogenic differentiation is significantly promoted compared with that of Zfp217 alone (FIG. 16A-FIG. 16B). Upon overexpression of Zfp217 on C3H10T1/2 cells, osteogenic differentiation is inhibited and lipogenic differentiation is promoted; when both Zfp217 and Zfp521 are overexpressed, osteogenic differentiation is promoted and lipogenic differentiation is inhibited as compared to overexpression of Zfp217 alone (FIG. 16C-FIG. 16D). This suggests that Zfp217 regulates MSC fate decision in part by modulating Zfp521 expression.

12. Alleviation of Bone Loss in Estrogen-Deficient Mice by Bone Marrow Injection of agomiR-503-5p/siZfp217

In order to study the therapeutic effect of targeted inhibition of BMSCs on osteoporosis caused by estrogen deficiency, agomiR-503-5p/siZfp217 is injected into ovariectomized mice through bone marrow twice a month for 3 months.

Among them, the aptamer sequence is:

(SEQ ID NO: 24)
5′-gaattcagtcggacagcgacggtgatatgtcaaggtcgtaggcacga
gtcagagggatggacgaatatcgtctccc-3′;

the sequence of miR-503-5p is:

sense (5′-3′)
(SEQ ID NO: 25)
UAGCAGCGGGAACAGUACUGCAG;
antisense (5-3′)
(SEQ ID NO: 26)
GCAGUACUGUUCCCGCUGCUAUU;

the sequence of siZfp217 is:

sense (5′-3′)
(SEQ ID NO: 27)
TCACATCAGCACCTATCTAAC;
and
antisense (5-3′)
(SEQ ID NO: 28)
GTTAGATAGGTGCTGATGTGA.

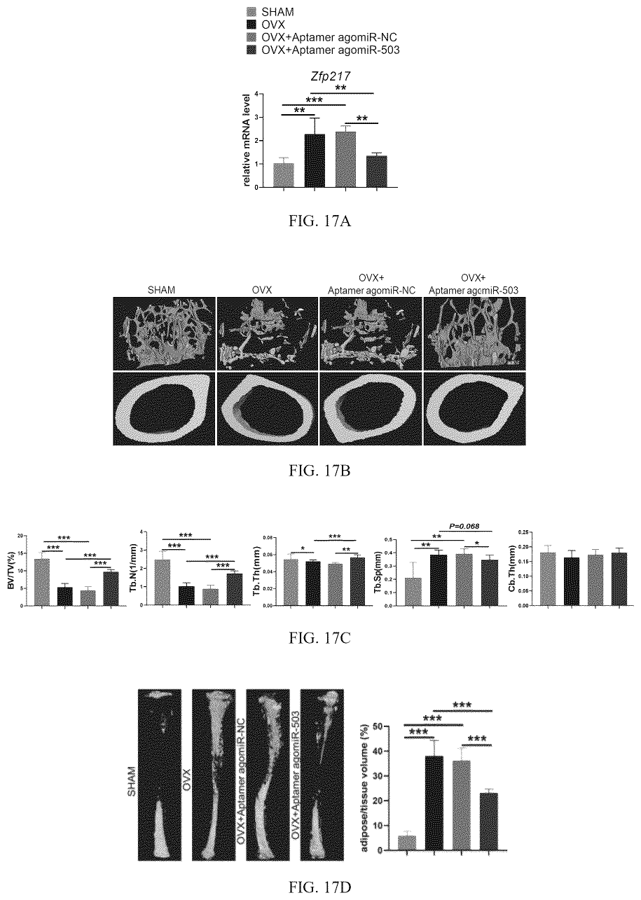

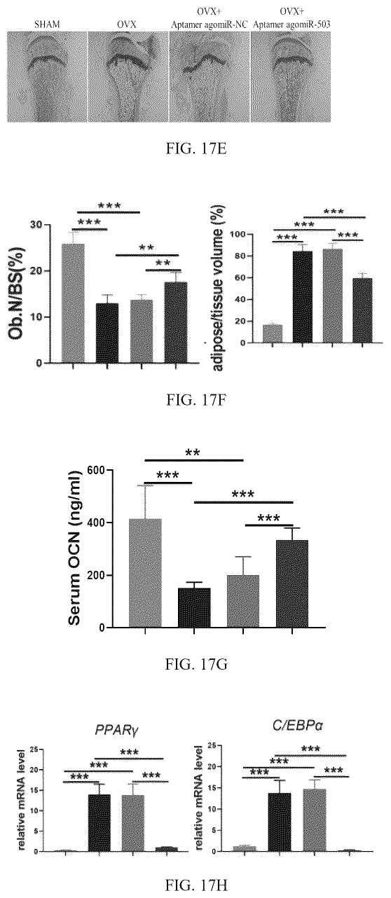

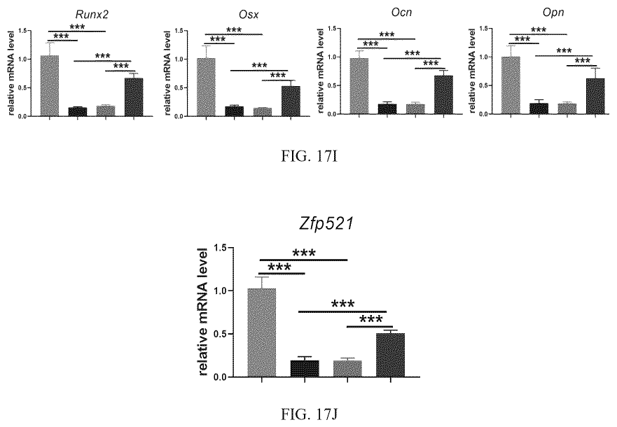

The level of Zfp217 in BMSCs is significantly decreased after the bone marrow injection of the aptamer agomir-503-5p/siZfp217 targeting BMSCs (FIG. 17A). The BV/TV Tb.N and Tb.Th are increased and Tb.Sp is decreased in mice injected with the aptamer-agomiR-503-5p/siZfp217 compared with de-ovulated mice (FIG. 17B-FIG. 17C). In addition, the area of adipocytes is reduced by injection of aptamer-agomiR-503-5p/siZfp217 after ovariectomy compared to WT-OVX mice (FIG. 17D). Besides, mice treated with aptamer-agomiR-503-5p/siZfp217 show a significant increase in the number of osteoblasts and a decrease in adipose volume (FIG. 17E-FIG. 17F). And, intramedullary injection of aptamer-agomiR-503-5p/siZfp217 increases serum OCN levels in OVX mice compared with WT-OVX mice (FIG. 17G). Furthermore, the aptamer-agomiR-503-5p/siZfp217 is found to significantly attenuate estrogen deficiency-induced decrease in osteogenic differentiation and to significantly reduce the increase in adipogenesis triggered by estrogen deficiency (FIG. 17H and FIG. 17I). Finally, the expression of Zfp521 is significantly increased in mice injected with aptamer-agomiR-503-5p/siZfp217 compared with WT-OVX (FIG. 17J). These results suggest that intramedullary injection of aptamer-agomiR-503-5p/siZfp217 prevents bone loss and bone marrow fat accumulation caused by estrogen deficiency.

### Embodiment 2

In this embodiment, a mouse with conditional knockout of BMSCs Zfp217 is used to construct a tail suspension model to simulate the situation of mechanical stress deficiency, and polyacrylamide gels with different hardness are used to simulate the cell environment in the state of stress deficiency to study the influence of mechanical stress on Zfp217. Finally, through the polyacrylamide model, the regulation mechanism of stress on Zfp217 and the role that Zfp217 plays in the fate selection process of BMSCs are studied, so as to further improve the role that Zfp217 plays in the process of bone formation.

The above-mentioned embodiments only describe the preferred mode of the present disclosure, and do not limit the scope of the present disclosure. Under the premise of not departing from the design spirit of the present disclosure, various modifications and improvements made by ordinary technicians in the field to the technical scheme of the present disclosure shall fall within the protection scope determined by the claims of the present disclosure.

What is claimed is:

1. A method for treating osteoporosis, comprising inhibiting an expression of a zinc finger protein 217 (Zfp217) gene and ameliorating osteoporosis symptoms.

2. The method according to claim 1, wherein the osteoporosis comprises osteoporosis caused by estrogen deficiency and osteoporosis caused by mechanical stress deficiency.

3. The method according to claim 1, wherein by inhibiting the expression of the Zfp217 gene, an expression of a zinc finger protein 521 (Zfp521) gene is promoted, thereby promoting a formation of osteoblasts.

4. The method according to claim 1, wherein by inhibiting the expression of the Zfp217 gene, bone marrow fat is reduced and bone mass is increased.

5. A method for regulating a differentiation of bone marrow mesenchymal stem cells into osteoblasts or adipocytes, comprising regulating an expression of a zinc finger protein 217 (Zfp217) gene, wherein the bone marrow mesenchymal stem cells differentiate into the osteoblasts when the expression of the Zfp217 gene is inhibited, and the bone marrow mesenchymal stem cells differentiate into the adipocytes when the expression of the Zfp217 gene is promoted.

6. A medicine for treating osteoporosis, wherein the medicine comprises a reagent for inhibiting an expression of a zinc finger protein 217 (Zfp217) gene.

7. The medicine according to claim 6, wherein the reagent for inhibiting the expression of the Zfp217 gene comprises an aptamer for inhibiting the expression of the Zfp217 gene in bone marrow mesenchymal stem cells.

8. The medicine according to claim 7, wherein the aptamer for inhibiting the expression of the Zfp217 gene in the bone marrow mesenchymal stem cells comprises a sequence shown in SEQ ID NO: 25.

9. The medicine according to claim 6, wherein the osteoporosis comprises osteoporosis caused by estrogen deficiency and osteoporosis caused by mechanical stress deficiency.


SEQUENCE LISTING (28 sequences)

SEQ ID NO: 1 - DNA, 18 bp, synthetic construct
  source 1..18: mol_type = other DNA
agaacggcagcagcaagt

SEQ ID NO: 2 - DNA, 20 bp, synthetic construct
  source 1..20: mol_type = other DNA
agtgtggtgagaaggcagtc

SEQ ID NO: 3 - DNA, 18 bp, synthetic construct
  source 1..18: mol_type = other DNA
agccaagaagtcggtaga

SEQ ID NO: 4 - DNA, 18 bp, synthetic construct
  source 1..18: mol_type = other DNA
cggtcattgtcactggtc

SEQ ID NO: 5 - DNA, 22 bp, synthetic construct
  source 1..22: mol_type = other DNA
tgggtgaaactctgggagattc

SEQ ID NO: 6 - DNA, 22 bp, synthetic construct
  source 1..22: mol_type = other DNA
agaggtccacagagctgattcc

SEQ ID NO: 7 - DNA, 20 bp, synthetic construct
  source 1..20: mol_type = other DNA
gccgggaatgatgagaacta

SEQ ID NO: 8 - DNA, 20 bp, synthetic construct
  source 1..20: mol_type = other DNA
ggaccgtccactgtcacttt

SEQ ID NO: 9 - DNA, 18 bp, synthetic construct
  source 1..18: mol_type = other DNA
gcaaggcttcgcatctga

SEQ ID NO: 10 - DNA, 20 bp, synthetic construct
  source 1..20: mol_type = other DNA
caagtggtcgcttctggtaa

SEQ ID NO: 11 - DNA, 20 bp, synthetic construct
  source 1..20: mol_type = other DNA
atggcttgaagaccgcctac

SEQ ID NO: 12 - DNA, 20 bp, synthetic construct
  source 1..20: mol_type = other DNA
agggcagagagagaggacag

SEQ ID NO: 13 - DNA, 23 bp, synthetic construct
  source 1..23: mol_type = other DNA
gacgatgatgacgatgatgatga

SEQ ID NO: 14 - DNA, 22 bp, synthetic construct
  source 1..22: mol_type = other DNA
tagggacgattggagtgaaagt

SEQ ID NO: 15 - DNA, 21 bp, synthetic construct
  source 1..21: mol_type = other DNA
ggcaccacaccttctacaatg

SEQ ID NO: 16 - DNA, 21 bp, synthetic construct
  source 1..21: mol_type = other DNA
ggggtgttgaaggtctcaaac

SEQ ID NO: 17 - DNA, 19 bp, synthetic construct
  source 1..19: mol_type = other DNA
ctcagcagacctccgatat

SEQ ID NO: 18 - DNA, 21 bp, synthetic construct
  source 1..21: mol_type = other DNA
gtaagacctccaagcaatact

SEQ ID NO: 19 - DNA, 29 bp, synthetic construct
  source 1..29: mol_type = other DNA
cgggatccatgccgacccagtccctcctc

SEQ ID NO: 20 - DNA, 36 bp, synthetic construct
  source 1..36: mol_type = other DNA
gatatctcaaggttttttgtcatttggtcggtaatg

SEQ ID NO: 21 - DNA, 21 bp, synthetic construct
  source 1..21: mol_type = other DNA
cacacttccacggaatcatac

SEQ ID NO: 22 - DNA, 21 bp, synthetic construct
  source 1..21: mol_type = other DNA
tcacatcagcacctatctaac

SEQ ID NO: 23 - DNA, 19 bp, synthetic construct
  source 1..19: mol_type = other DNA
ttctccgaacgtgtcacgt

SEQ ID NO: 24 - DNA, 76 bp, synthetic construct
  source 1..76: mol_type = other DNA
gaattcagtcggacagcgacggtgatatgtcaaggtcgtaggcacgagtcagagggatggacgaatatcgtctccc

SEQ ID NO: 25 - DNA, 23 bp, synthetic construct
  source 1..23: mol_type = other DNA
tagcagcgggaacagtactgcag

SEQ ID NO: 26 - DNA, 23 bp, synthetic construct
  source 1..23: mol_type = other DNA
gcagtactgttcccgctgctatt

SEQ ID NO: 27 - DNA, 21 bp, synthetic construct
  source 1..21: mol_type = other DNA
tcacatcagcacctatctaac

SEQ ID NO: 28 - DNA, 21 bp, synthetic construct
  source 1..21: mol_type = other DNA
gttagataggtgctgatgtga